(19) United States
(12) Patent Application Publication
(10) Pub. No.: US 2023/0349571 A1
(43) Pub. Date: Nov. 2, 2023

(54) AIR PURIFICATION EQUIPMENT AND METHOD

(71) Applicant: Chiun Mai Communication Systems, Inc., New Taipei (TW)

(72) Inventors: CHIU-JU LIN, New Taipei (TW); FU-HSIN SUNG, New Taipei (TW); CHIA-JUNG HU, New Taipei (TW); MENG-YU CHOU, New Taipei (TW)

(21) Appl. No.: 18/128,211

(22) Filed: Mar. 29, 2023

(30) Foreign Application Priority Data
Mar. 31, 2022 (CN) 202210346723.1

(51) Int. Cl. A61L 9/20; F24F 8/22

(52) U.S. Cl. CPC F24F 8/22; A61L 9/20; F24F 2110/30

(57) ABSTRACT

An air purification equipment and an air purification method are provided, including a housing, a purification device, a guiding assembly, and an air driving device. The housing defines an air inlet and an air outlet, the air inlet imports air into the housing, the air outlet exports air out of the housing. The purification device purifies the air in the housing. The guiding assembly guides the air from the air inlet to the purification device. The air driving device drives the air from the air inlet to the purification device, and drives the air to the air outlet. The guiding assembly forms a first channel and a second channel, the first channel is corresponding to the air inlet, the first channel guides the air to flow into the second channel, at least a part of the purification device is positioned in the second channel.

### CROSS-REFERENCE TO RELATED APPLICATIONS

This application claims priority to Chinese Patent Application No. 202210346723.1 filed on Mar. 31, 2022, in China National Intellectual Property Administration, the contents of which are incorporated by reference herein.

### FIELD

The subject matter herein generally relates to air purification technologies, and specially relates to an air purification equipment and an air purification method.

### BACKGROUND

For improving indoor environment, air purification equipment may be mounted indoor. The air purification equipment may include a purification module, the air purification equipment may inhale partial air into an internal of the air purification equipment, after purification by the purification module, the purified air may be exhaled outside the air purification equipment, so as to purifies air of the indoor environment.

For improving an air purification efficiency, the air purification equipment has a high inhale and exhale speed, which may cause a time period of the air stays inside the air purification equipment is not long enough and a poor air purification effect.

### BRIEF DESCRIPTION OF THE DRAWINGS

Implementations of the present disclosure will now be described, by way of example only, with reference to the attached figures.

FIG. 1 is a schematic diagram of at least one embodiment of an air purification equipment showing internal structure.

FIG. 2 is a schematic diagram of at least one embodiment of a purification device of the air purification equipment.

FIG. 3 is another schematic diagram of at least one embodiment of the air purification equipment showing internal structure.

FIG. 4 is a partial cross-sectional diagram of at least one embodiment of a chassis.

FIG. 5 is another schematic diagram of at least one embodiment of the air purification equipment showing internal structure.

FIG. 6 is an explored diagram of at least one embodiment of the air purification equipment.

FIG. 7 is a schematic diagram of at least one embodiment of a system of the air purification equipment.

FIG. 8 is a flow chat of at least one embodiment of an air purification method.

FIG. 9 is another flow chat of at least one embodiment of the air purification method.

FIG. 10 is another flow chat of at least one embodiment of the air purification method.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better show details and features of the present disclosure.

Several definitions that apply throughout this disclosure will now be presented.

The term “coupled” is defined as connected, whether directly or indirectly through intervening components, and is not necessarily limited to physical connections. The connection may be such that the objects are permanently connected or releasably connected. The term “substantially” is defined to be essentially conforming to the particular dimension, shape, or other feature that the term modifies, such that the component need not be exact. For example, “substantially cylindrical” means that the object resembles a cylinder, but may have one or more deviations from a true cylinder. The term “comprising,” when utilized, means “including, but not necessarily limited to”; it specifically indicates open-ended inclusion or membership in the so-described combination, group, series, and the like.

The present disclosure is described in relation to an air purification equipment and a wearable device having the same.

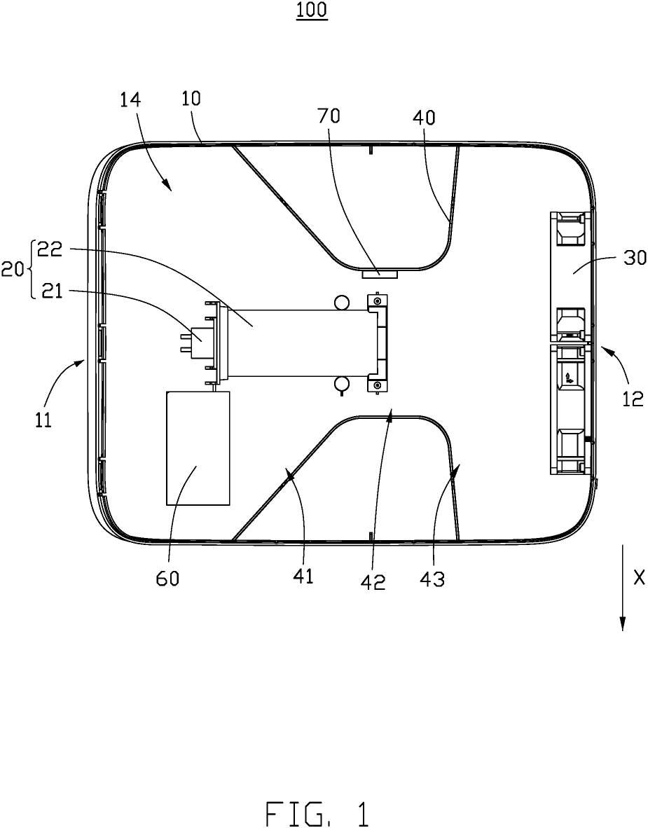

FIG. 1 illustrates a schematic diagram of at least one embodiment of an air purification equipment 100 showing internal structure. The air purification equipment 100 is configured to purify air.

In at least one embodiment, the air purification equipment 100 includes a housing 10. An end of the housing 10 defines an air inlet 11, another end of the housing 10 defines an air outlet 12. Each of the air inlet 1 1 and the air outlet 12 communicates an inside and an outside of the housing 10. The housing 10 arranges a purification device 20 therein. The purification device 20 is positioned between the air inlet 11 and the air outlet 12 and configured to purify air in the housing 10. The air inlet 11 is configured to import air to be purified into the housing 10. The air outlet 12 is configured to export purified air out of the housing 10.

In at least one embodiment, the air purification equipment 100 may be arranged indoor or on a ventilation opening connecting the indoor and outdoor. Indoor air or outdoor air may enter the housing 10 through the air inlet 11. The purification device 20 purifies the air in the housing 10. The purified air may be exported out of the housing 10 and enter the indoor environment. The air purified by the purification device 20 is not limited to air or gas.

For instance, the housing 10 may be a rectangular box having two short side inwalls and two long side inwalls. The air inlet 11 may be defined in one of the two short side inwalls, the air outlet 12 may be defined in the other one of the two short side inwalls.

In at least one embodiment, there may be a plurality of air inlets 11 defined on the housing 10 at intervals, and there may be a plurality of air outlets 12 defined on the housing 10 at intervals.

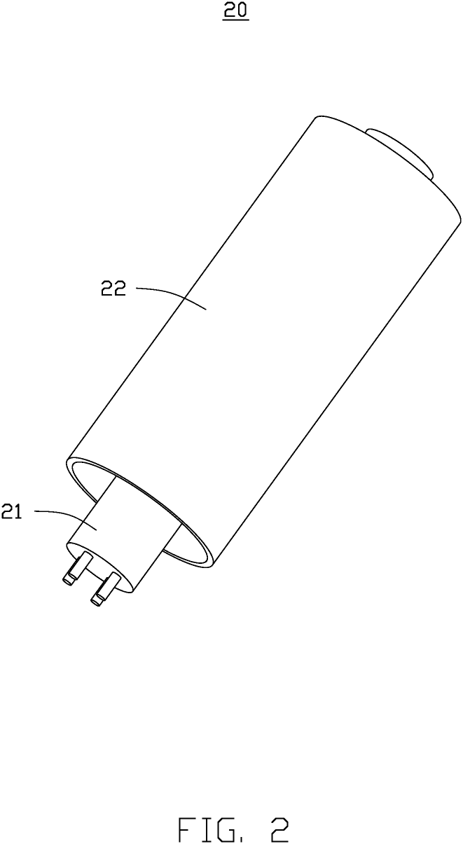

Referring to FIG. 2, in at least one embodiment, the purification device 20 may purify and disinfect the air using ultraviolet (UV) light. The purification device 20 includes a lamp tube 21 for emitting UV light. The lamp tube 21 may be secured to a supporter (not shown) on the inwall of the housing 10, so the lamp tube 21 may be secured to the inwall of the housing 10. The lamp tube 21 may be secured to the supporter through latching, which is not limited.

In at least one embodiment, the housing 10 may arrange an air driving device 30 therein. The air driving device 30 may be secured to the inwall of the housing 10. The air driving device 30 is positioned close to the air inlet 12. The air driving device 30 is configured to drive the air from the air inlet 11 to the purification device 20 for being purified, and drive the purified air from the purification device 20 to the air outlet 12 for being exported out of the housing 10.

The air driving device 30 may execute a one-way flow from the air inlet 11 to the air outlet 12, for decreasing running off the purified air from the air inlet 11.

For instance, the air driving device 30 may be one or more fan. The air driving device 30 may be secured to the inwall of the housing 10 defining the air outlet 12 through screws securing, which is not limited.

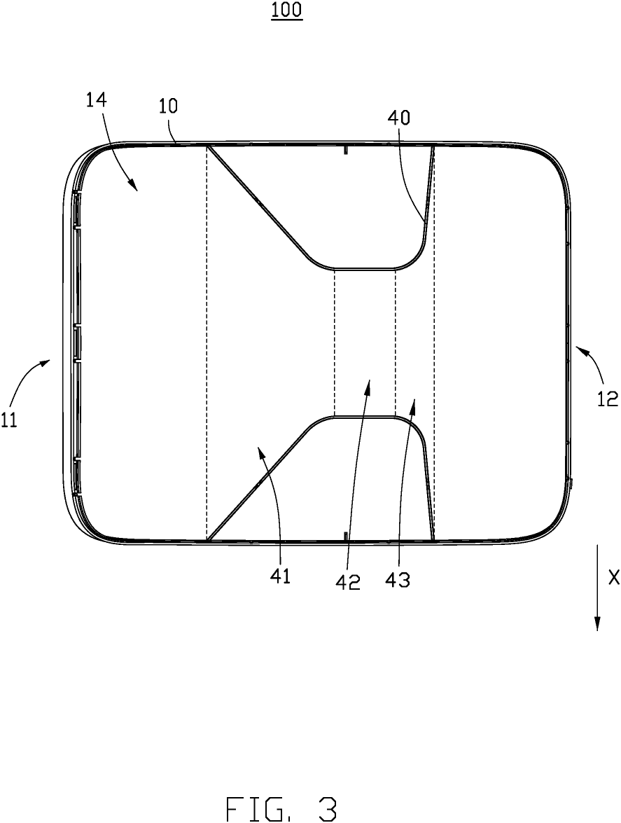

Referring to FIGS. 1 to 3, in at least one embodiment, the housing 10 further arranges a guiding assembly 40 between the air inlet 11 and the air outlet 12. The guiding assembly 40 is configured to guide the air from the air inlet 11 to the purification device 20 for being purified.

The guiding assembly 40 may be formed by extending on the inwall of the housing 10, or secured to the inwall of the housing 10 by joint structures, such as sticking, bonding, screws, etc.

In at least one embodiment, the guiding assembly 40 may form a first channel 41 and a second channel 42. The first channel 41 and the second channel 42 are communicated with each other. The first channel 41 is corresponding to the air inlet 11. The second channel 42 is positioned on a side of the first channel 41 away from the air inlet 11. A cross-sectional area of the first channel 41 is gradually decreasing along a direction from the air inlet 11 towards the air outlet 12. A cross-sectional area of the second channel 42 is smaller than a cross-sectional area of the first channel 41 closes to the air inlet 11, the cross-sectional area of the second channel 42 is a same along a direction from the air inlet 11 towards the air outlet 12. At least a part of the lamp tube 21 is positioned in the second channel 42. There is a space in a width direction of the housing 10 between the lamp tube 21 and the part of the guiding assembly 40 forming the second channel 42. The first channel 41 is configured to guide air into the second channel 42, so the air may be purified in a smaller space with the lamp tube 21 for improving a purifying effect.

In at least one embodiment, the lamp tube 21 may be fully positioned in the second channel 42. In another embodiment, the lamp tube 21 may be partially positioned in the second channel 42, and the rest of the lamp tube 21 may be positioned in the first channel 41. In another embodiment, the lamp tube 21 may be partially positioned in the second channel 42, and the rest of the lamp tube 21 may be extended out of the second channel 42 from a direction away from the first channel 41, which is not limited by the present disclosure.

A width direction of the first channel 41, a width direction of the second channel 42, and a width direction of the housing 10 may be a same direction, such as a X direction shown in FIG. 1. The width direction of the housing 10 may be a length of the short side inwall of the housing 10.

The housing 10 may arrange two guiding assemblies 40 at interval along the width direction of the housing 10. The two guiding assemblies 40 are symmetry. Each of the two guiding assemblies 40 is substantially bent board shaped, the two symmetric guiding assemblies 40 can form the first channel 41 of a funnel shaped and the second channel 42 of a rectangular shaped. Both the first channel 41 and the second channel 42 are symmetric.

A width of the second channel 42 is smaller than a width of the housing 10, a cross-sectional area of the second channel 42 is smaller than a cross-sectional area of the housing 10. A width of a portion of the first channel 41 closes to the air inlet 11 may be smaller than or equal to the width of the housing 10, a cross-sectional area of the portion of the first channel 41 closes to the air inlet 11 may be smaller than or equal to the cross-sectional area of the housing 10, which is not limited by the present disclosure.

A cross-sectional area of a portion of the first channel 41 facing the air inlet 11 is greater than a cross-sectional area of a portion of the first channel 41 facing the air outlet 12. In at least one embodiment, two side walls of the firs channel 41 are arranged on the two long side inwalls of the housing 10, air enters the first channel 41 from the air inlet 11 may flow along the side walls of the firs channel 41, and form a turbulent flow in an end of the first channel 41 that away from the second channel 42. When the air driving device 30 drives the air to flow, which may synchronously improve a flow speed in the end of the first channel 41 that away from the second channel 42. Air flows from the first channel 41 to a connection of the first channel 41 and the second channel 42, and then flows into the second channel 42. When air flows to the second channel 42, air can be illuminated by the UV light by short distance in the second channel 42. The arrangement of the first channel 41 and the second channel 42 may increase a time period that the air stays close to the lamp tube 21, so the air can stay longer for being illuminated by the UV light, which improves an air purifying effect.

The inwalls of the first channel 41 may be substantially planar, cambered, or stepped plane, which is not limited.

In a first embodiment, a cross-sectional area of a portion of the first channel 41 away from the air inlet 11 is equal to a cross-sectional area of the second channel 42. In a second embodiment, the cross-sectional area of the portion of the first channel 41 away from the air inlet 11 is greater than the cross-sectional area of the second channel 42. In a third embodiment, the cross-sectional area of the portion of the first channel 41 away from the air inlet 11 is smaller than the cross-sectional area of the second channel 42, which is not limited, the first embodiment is used for the following description.

In other embodiments, the guiding assembly 40 further forms a third channel 43. The third channel 43 and the first channel 41 are positioned on opposite sides of the second channel 42. The third channel 43 connects the second channel 42. The third channel 43 is corresponding to the air outlet 12. A cross-sectional area of the third channel 43 is gradually increasing along the direction from the air inlet 11 towards the air outlet 12. The third channel 43 is configured to guide and accelerate purified air in the second channel 42 to the air outlet 12, so as to guide purified air out of the housing 10.

The two symmetric guiding assemblies 40 can form the third channel 43 of a funnel shaped. A cross-sectional area of a portion of the third channel 43 closes to the air outlet 12 is smaller than or equal to the cross-sectional area of the housing 10, which is not limited.

In a first embodiment, the purification device 20 may be fully positioned in the second channel 42. In a second embodiment, the purification device 20 may be partially positioned in the first channel 41, and the rest of the purification device 20 may be positioned in the second channel 42. In a third embodiment, an end of the purification device 20 may be positioned in the first channel 41, another end of the purification device 20 may be positioned in the third channel 43, a middle portion of the purification device 20 may be positioned in the second channel 42, which is not limited.

The inwalls of the third channel 43 may be substantially planar, cambered, or stepped plane, which is not limited.

In at least one embodiment, the air driving device 30 drives the purified air in the second channel 42 to flow towards the third channel 43, and drives the purified air to flow from the third channel 43 to the air outlet 12.

A cross-sectional area of a portion of the third channel 43 away from the air outlet 12 is equal to the cross-sectional area of the second channel 42. In a second embodiment, the cross-sectional area of the portion of the third channel 43 away from the air outlet 12 is greater than the cross-sectional area of the second channel 42. In a third embodiment, the cross-sectional area of the portion of the third channel 43 away from the air outlet 12 is smaller than the cross-sectional area of the second channel 42, which is not limited, the first embodiment is used for the following description.

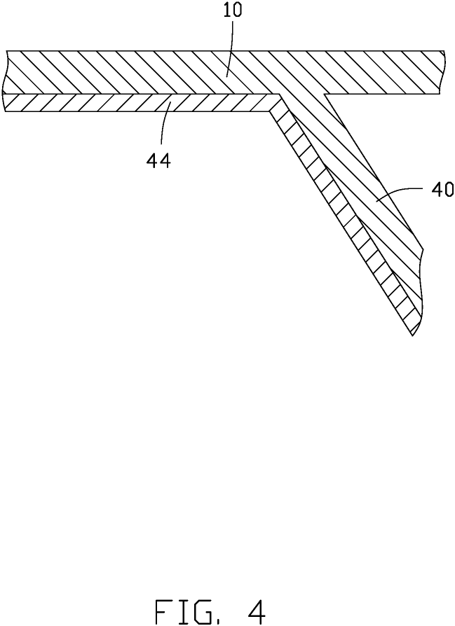

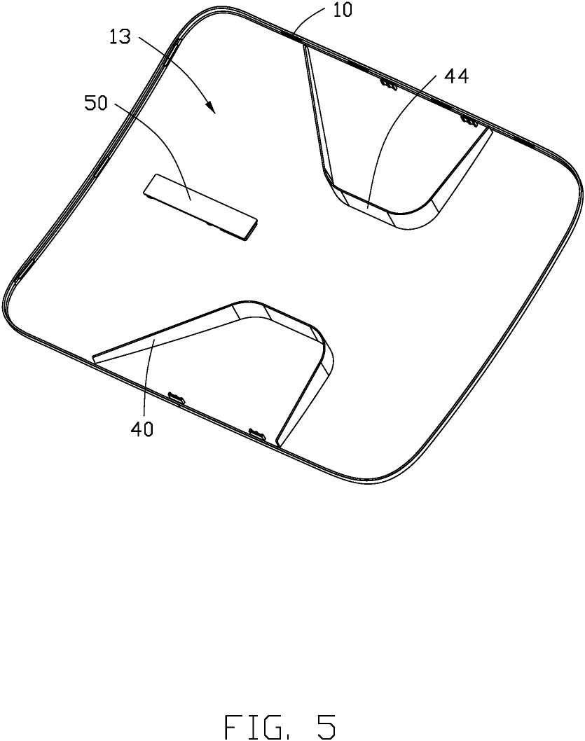

Referring to FIGS. 4 and 5, in at least one embodiment, a surface of each of the two guiding assemblies 40 facing to each other is arranged with a reflector 44. The reflector 44 is configured to reflect the UV light emitted by the lamp tube 21 to decrease absorb of the UV light by the guiding assemblies 40, which may improve an illumination of the UV light in the housing 10 and a purifying effect.

The reflector 44 can be but is not limited to copper foil. A secured way between the reflector 44 and the guiding assembly 40 can be but is not limited to sticking.

In at least one embodiment, the inwalls of the housing 10 may be arranged with the reflector 44. The reflector 44 arranged on the guiding assemblies 40 and the reflector 44 arranged on the inwalls of the housing 10 may be made of same materials. A structure of the reflector 44 secured to the guiding assemblies 40 and a structure of the reflector 44 secured to the inwalls of the housing 10 may be the same.

Referring to FIGS. 1 and 2, in at least one embodiment, the purification device 20 may further include a fiberglass sleeve 22 covering on the lamp tube 21 for protecting the lamp tube 21 and increasing a medium besides the air for transmitting the UV light.

The UV light may pass through the fiberglass sleeve 22 after entering the fiberglass sleeve 22, since a scatter ratio of light in the fiberglass is smaller than a scatter ratio of light in the air, the fiberglass sleeve 22 may decrease the scatter ratio of the UV light in the housing 10, which may improve an illumination of the UV light in the housing 10 and a purifying effect.

The fiberglass sleeve 22 may be secured to a securing structure arranged on the inwalls of the housing 10, which may be sticking, latching, etc.

In at least one embodiment, the fiberglass sleeve 22 includes photocatalyst purification factors, which can degrade harmful air, such as formaldehyde, in the air under the illumination of the UV light, for improving a purifying effect.

In at least one embodiment, the photocatalyst purification factors may be painted on the fiberglass sleeve 22. In other embodiments, the photocatalyst purification factors may be a part of the materials that the fiberglass sleeve 22 made of, which is not limited.

In at least one embodiment, the air purification equipment 100 may further include a processor 60. The processor 60 is configured to output notification information according to detected information for noticing a user of a working status of the air purification equipment 100.

The processor 60 may be a central processing unit (CPU), or may be another general-purpose processor, a digital signal processor (DSP), an application specific integrated circuit (ASIC), a Field-Programmable Gate Array (FPGA) or other programmable logic devices, discrete gates or transistor logic devices, discrete hardware components, or the like.

The detected information may be information detected by an electronic device having environment detection function and obtained by the processor 60, or information obtained by the processor 60 from other elements of the air purification equipment 100, which is not limited.

The processor 60 may analyzes the detected information to determine the working status of the air purification equipment 100, and output notification information corresponding to the working status of the air purification equipment 100 to notice the user the working status of the air purification equipment 100. The processor 60 outputs different notification information according to different working status of the air purification equipment 100.

In at least one embodiment, the processor 60 may be electrically connected to a user equipment (not shown) through a communication device, the processor 60 outputs notification information to the user equipment showing the working status of the air purification equipment 100 for being browsed by the user. The user equipment may be an interactive device having a displaying function for displaying image and text information and communicating function. The user equipment may be but is not limited to a mobile phone, a computer, a tablet computer, a personal digital assistant, a wearable device, a smart bracelet, a smart glasses, etc. The notification information may in a form of e-mail, notification of an application, text message, etc.

For instance, when the processor 60 determines an illuminance of the lamp tube 21 is within a predetermined range according to the detected information, the processor 60 outputs notification information corresponding to the illuminance of the lamp tube 21 to the user equipment, the notification information may be a text message showing the illuminance of the lamp tube 21.

In other embodiments, the air purification equipment 100 may include an alarm device (not shown), the processor 60 may be electrically connected to the alarm device, the alarm device may output alarm information in response to the notification information. For instance, the alarm device may be an alarm lamp and/or an audio player.

For instance, the alarm device may include alarm lamps, when the processor 60 determines a flow velocity of the air in the housing 10 is within a predetermined range according to the detected information, the processor 60 outputs notification information corresponding to the flow velocity of the air in the housing 10 to the alarm device, the alarm device outputs alarm information according to the notification information, an alarm lamp of the alarm device corresponding to a tag of normal air flow velocity turns on.

In at least one embodiment, the detected information may include light detected information, the notification information may include illuminance notification information. The air purification equipment 100 may further include a light sensor 50 electrically connected to the processor 60. The light sensor 50 is configured to detect an illuminance of the UV light emitted by the lamp tube 21 and output light detected information to the processor 60, the processor 60 outputs illuminance notification information according to the light detected information.

The light sensor 50 has a UV light detecting function and is capable of converting light signals into electric signals. The light sensor 50 can be but is not limited to a light sensor.

The light sensor 50 and the processor 60 can be in wireless communication or connected in cables, which is not limited.

Before the lamp tube 21 turns on, a standard illuminance in the housing 10 can be determined according to parameters of the lamp tube 21. When the light sensor 50 detects an illuminance emitted in the housing 10 and outputs light detected information to the processor 60, the processor 60 analyzes the light detected information to determine the illuminance in the housing 10. If there is a same or a difference within a predetermined range between the determined illuminance and the standard illuminance, the lamp tube 21 can work normally is determined. If there is a difference without the predetermined range between the determined illuminance and the standard illuminance, the lamp tube 21 being abnormal is determined, the processor 60 then outputs the illuminance notification information.

In at least one embodiment, the processor 60 can output the illuminance notification information to the user equipment to notice the user that the lamp tube 21 needs to be repaired.

A content of the illuminance notification information outputted by the processor 60 to the user equipment can be the illuminance in the housing 10, the illuminance in the housing 10 can be generated by the processor 60 through analyzing the light detected information. The content of the illuminance notification information outputted by the processor 60 to the user equipment can also be texts of lamp tube abnormal or purification device abnormal.

In other embodiments, the processor 60 can be electrically connected to the alarm device, the alarm device outputs alarm information in response to the illuminance notification information. For instance, alarm device may be an alarm lamp and/or an audio player.

Referring to FIGS. 1 to 5, in at least one embodiment, the housing 10 includes a top wall 13 and a bottom wall 14. The purification device 20 is mounted on the bottom wall 14, the light sensor 50 is mounted on the top wall 13 and corresponding to the purification device 20. The light sensor 50 is configured to detect the illuminance of the UV light emitted by the lamp tube 21 in the housing 10.

The light sensor 50 may by secured to the top wall 13 by screws.

Referring to FIG. 1, in at least one embodiment, the processor 60 may be mounted on the bottom wall 14 and positioned between the air inlet 11 and the purification device 20. Since the air in the housing 10 keeps flowing from the air inlet 11 to the air outlet 12, the air flow may decrease a heat transmission from the heat generated by the lamp tube 21 to the air inlet 11 and a heat transmission from the heat generated by the lamp tube 21 to the processor 60. Meanwhile, the air flow may dissipate heat from the processor 60 and out of the housing 10 through the air outlet 12, which may lengthen a usage of the processor 60.

The processor 60 may by secured to the bottom wall 14 of the housing 10 by screws.

The processor 60 may be integrated with other electronic elements of the air purification equipment 100 and mounted between the air inlet 11 and the purification device 20.

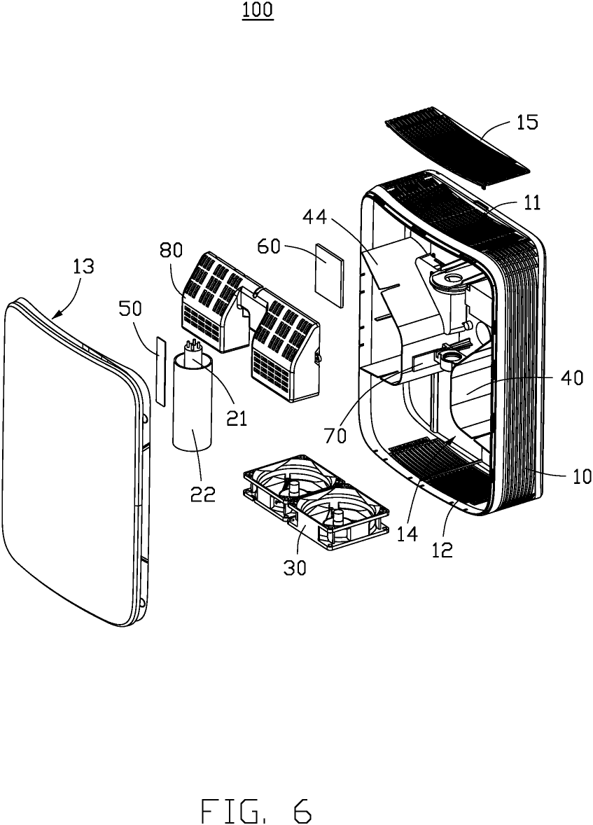

Referring to FIG. 6, in at least one embodiment, the housing 10 arranges with a protection cover 80 therein. The protection cover 80 covers the processor 60 and other electronic elements. The protection cover 80 may be fixedly connected to the inwalls of the housing 10. The protection cover 80 defines a plurality of holes for the air flow. The protection cover 80 is configured to decrease an illuminance area of the UV light illuminates on the processor 60 and other electronic elements, which may lengthen the usage of the processor 60 and other electronic elements.

The protection cover 80 may by secured to the inwalls of the housing 10 by screws.

In at least one embodiment, the detected information further includes air flow velocity information, the notification information further includes air flow velocity abnormal notification information. The processor 60 may determine whether the air flow velocity in the housing 10 is within a predetermined range according to the air flow velocity information, when the air flow velocity in the housing 10 is without the predetermined range, the processor 60 outputs air flow velocity abnormal notification information to notice the user that the air inlet 11 may be blocked or stopped, which needs cleaning.

In at least one embodiment, the air flow velocity information may be driving current of the air driving device 30. The processor 60 is electrically connected to the air driving device 30. When the air driving device 30 is switched on, the processor 60 may obtain the driving current of the air driving device 30 and determine whether the driving current is within a predetermined range. When the driving current is without the predetermined range, the processor 60 outputs the air flow velocity notification information.

The processor 60 may output the air flow velocity notification information to the alarm device or the user equipment, which is not limited.

The air driving device 30 may maintain a stable air flow velocity, when the air inlet 11 is sticked with dust that blocks the air entering the housing 10, the air driving device 30 can increase the driving current and/or power through an adaptive program, to increase a driving force to the air flow. The processor 60 may determine whether the driving current of the air driving device 30 is within the predetermined range, to determine whether the air inlet 11 is blocked. When the air inlet 11 being blocked is determined, the processor 60 outputs the air flow velocity notification information to notice the user that the air inlet 11 needs cleaning.

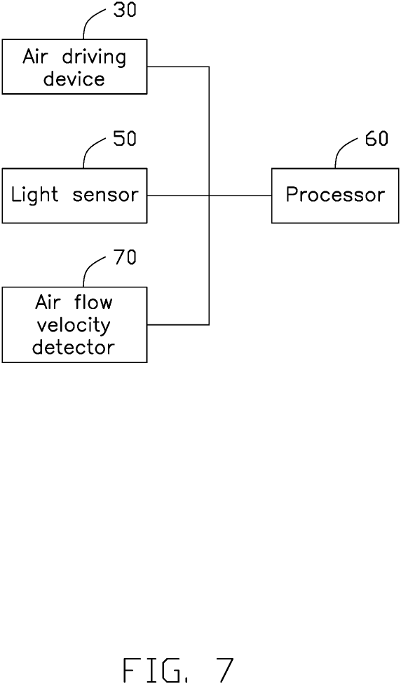

Referring to FIGS. 6 and 7, in at least one embodiment, the air purification equipment 100 may further include an air flow velocity detector 70 secured to the inwalls of the housing 10. The air flow velocity detector 70 is electrically connected to the processor 60. The air flow velocity detector 70 is configured to detect air flow velocity in the housing 10 and output air flow velocity information to the processor 60, the processor 60 analyzes the air flow velocity information to determine the air flow velocity in the housing 10. When the air flow velocity in the housing 10 is smaller than a predetermined threshold value, the processor 60 may output air flow velocity notification information to notice the user that the air inlet 11 needs cleaning. The predetermined threshold value may be a maximum value of the predetermined range of air flow velocity in the housing 10.

In at least one embodiment, the air flow velocity detector 70 may be mounted on a side portion, a top portion, or bottom portion of the lamp tube 21. For instance, the air flow velocity detector 70 may be mounted on the guiding assembly 40, such as inwalls of the second channel 42.

The air flow velocity detector 70 may be mounted on the guiding assembly 40 through screws or sticking.

When the driving current of the air driving device 30 is constant, any block in the air inlet 11 may decrease the air flow velocity in the housing 10. When the air flow velocity in the housing 10 is smaller than the predetermined threshold value, the air purification equipment 100 may has a weaker purification effect to the indoor air, so the processor 60 may output air flow velocity notification information to notice the user that the air inlet 11 needs cleaning.

The air flow velocity detector 70 and the processor 60 can be in wireless communication or connected in cables, which is not limited.

External walls of the housing 10 may detachably arrange with a filter 15. The filter 15 may be secured to the air inlet 11. When the processor 60 outputs the air flow velocity notification information, the filter 15 may be disassembled from the housing 10 by the user.

The filter 15 may block dust from the housing 10, when the air flow velocity notification information is outputted, cleaning the filter 15 is only needed, and the housing 10 does not need to be cleaned, which improve a cleaning convenience.

The filter 15 and the housing 10 may be connected through screws or latching, which is not limited.

In at least one embodiment, when the air purification equipment 100 is switched on, the air driving device 30 drives air flowing into the housing 10 through the air inlet 11 and the air in the housing 10 flowing towards the air outlet 12, the guiding assemblies 40 guide the air flowing into the first channel 41 and the second channel 42, the purification device 20 emits UV light to purify the air. The first channel 41 guides the air to stay longer in the second channel 42 for longer purification. The fiberglass sleeve 22 and the reflector 44 improve the UV light illuminance in the housing 10, especially where closes to the lamp tube 21, to improve the purifying effect to the air. The purified air enters the third channel 43, along with the cross-sectional area of the third channel 43 is gradually increasing, the air flow velocity of the purified air increases, the purified air then is driven by the air driving device 30 to flow out of the housing 10.

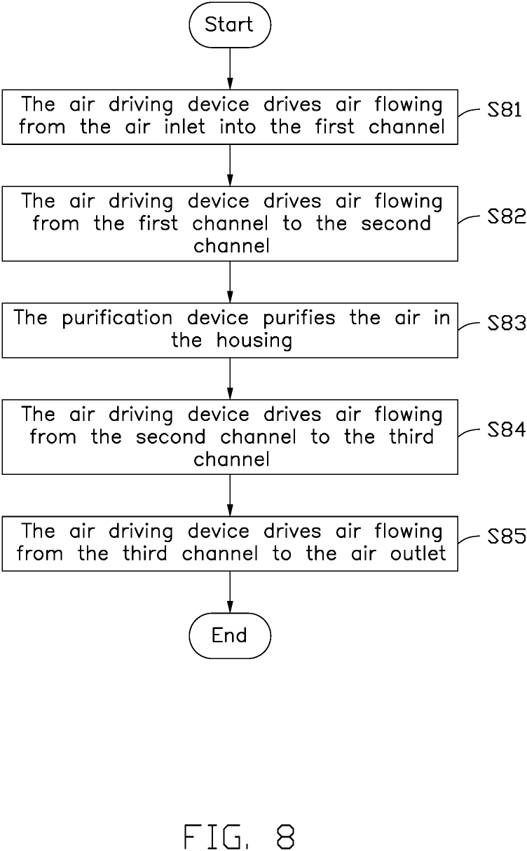

FIG. 8 illustrates a flow chat of at least one embodiment of an air purification method, which may be applied in the air purification equipment 100. The air purification method may include:

At block S81, the air driving device 30 drives air flowing from the air inlet 11 into the first channel 41.

At block S82, the air driving device 30 drives air flowing from the first channel 41 to the second channel 42.

The first channel 41 may guide the air flowing into the second channel 42.

At block S83, the purification device 20 purifies the air in the housing 10.

In at least one embodiment, the purification device 20 is fully positioned in the second channel 42, the air stays in the second channel 42 may be purified by long-time and short-distance illumination of the lamp tube 21, which improves a purifying effect. In another embodiment, the purification device 20 is partially positioned in the first channel 41, and the rest of the purification device 20 is positioned in the second channel 42. In another embodiment, an end of the purification device 20 is positioned in the first channel 41, another end of the purification device 20 is positioned in the third channel 43, a middle portion of the purification device 20 is positioned in the second channel 42, which is not limited.

At block S84, the air driving device 30 drives air flowing from the second channel 42 to the third channel 43.

The air driving device 30 may drive the purified air or air under purifying flowing from the second channel 42 to the third channel 43 to make the air close to the air outlet 12.

At block S85, the air driving device 30 drives air flowing from the third channel 43 to the air outlet 12.

In at least one embodiment, when driving the air out of the second channel 42, the purified air may be driven by the air driving device 30 to rapidly flow out of the housing 10. In other embodiments, the guiding assembly 40 further forms the third channel 43 connecting to the second channel 42, the third channel 43 and the first channel 41 are positioned on opposite sides of the second channel 42, the third channel 43 is corresponding to the air outlet 12, the air driving device 30 drives the purified air out of the housing 10.

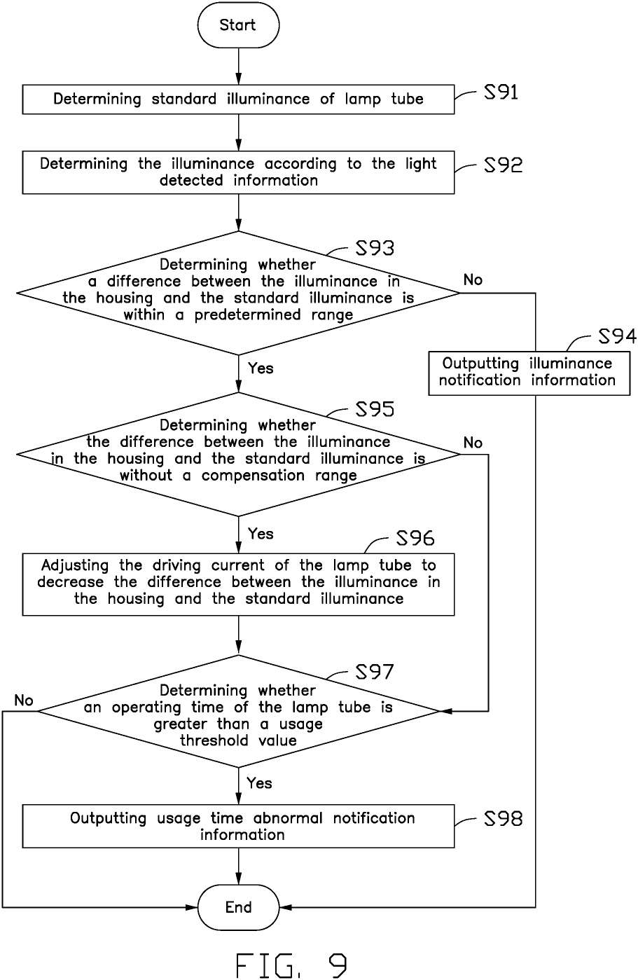

FIG. 9 illustrates another flow chat of at least one embodiment of the air purification method, the air purification method may be executed by the processor 60:

At block S91, determining a standard illuminance of the lamp tube 21.

The standard illuminance is corresponding to the illuminance of the UV light generated by the lamp tube 21 operated in a predetermined driving voltage and a predetermined driving current. Since the predetermined driving voltage and the predetermined driving current are known, the standard illuminance can be obtained.

In at least one embodiment, the processor 60 can be electrically connected to a data base (not shown), the processor 60 can obtain a working current value of the lamp tube 21, the data base stores rated illuminances corresponding to different working current values, the processor 60 can determine a rated illuminance corresponding to the obtained working current value in the data base, the rated illuminance corresponding to the obtained working current value can be the standard illuminance. In other embodiments, the standard illuminance may be set by the user after detecting the working current value of the lamp tube 21, such as by calculating or comparing the working current value to the rated illuminances in a specification of the lamp tube 21, then the user can output the standard illuminance to the processor 60 through the user equipment, the processor 60 determines the rated illuminance as the standard illuminance, which is not limited.

At block S92, determining the illuminance according to the light detected information.

The light sensor 50 detects an illuminance in the housing 10 and generates light detected information corresponding to the illuminance in the housing 10, the processor 60 obtains and analyzes the light detected information to determine the illuminance in the housing 10.

At block S93, determining whether a difference between the illuminance in the housing 10 and the standard illuminance is within a predetermined range, if the difference is without the predetermined range, the procedure goes to block S94; if the difference is within the predetermined range, the procedure goes to block S95, the illuminance in the housing 10 is corresponding to the light detected information.

When the difference between the illuminance in the housing 10 and the standard illuminance is within the predetermined range, the difference between the illuminance in the housing 10 and the standard illuminance may be overcame by adjusting the driving current of the lamp tube 21 to make the illuminance in the housing 10 closes to or equaled to the standard illuminance is determined, then the air purifying effect may be close to or equaled to an expected effect is determined. When the difference between the illuminance in the housing 10 and the standard illuminance is without the predetermined range, the lamp tube 21 being abnormal is determined.

At block S94, outputting illuminance notification information.

When the difference between the illuminance in the housing 10 and the standard illuminance is great, outputting illuminance notification information to notice the user that the lamp tube 21 needs to be repaired or changed.

In at least one embodiment, the processor 60 may output the illuminance notification information to the user equipment to notice the user that the lamp tube 21 needs to be repaired or changed. When processor 60 may output the illuminance notification information to the user equipment, the illuminance notification information may be in a form of e-mail, notification of applications, messages, etc.

A content of the illuminance notification information outputted from the processor 60 to the user equipment may show the illuminance in the housing 10, the illuminance in the housing 10 may be generated by the processor 60 after analyzing the light detected information. The content of the illuminance notification information outputted from the processor 60 to the user equipment may show text of “lamp tube illuminance abnormal” or “purification device illuminance abnormal”.

In another embodiment, the processor 60 may be electrically connected to the alarm device, the alarm device outputs alarm information in response to the illuminance notification information.

At block S95, determining whether the difference between the illuminance in the housing 10 and the standard illuminance is without a compensation range, if the difference is without the compensation range, the procedure goes to block S96; if the difference is within the compensation range, the procedure goes to block S97.

If the difference between the illuminance in the housing 10 and the standard illuminance is within the compensation range, the difference may has a low affection to the air purifying effect is determined, which may not need extract operations; if the difference between the illuminance in the housing 10 and the standard illuminance is without the compensation range, rated driving current of the lamp tube 21 may need compensated to strengthen the air purifying effect.

At block S96, adjusting the driving current of the lamp tube 21 to decrease the difference between the illuminance in the housing 10 and the standard illuminance, then the procedure goes to block S97.

The processor 60 may be electrically connected to the lamp tube 21 to adjust the driving current of the lamp tube 21. A method for adjust the driving current of the lamp tube 21 may include determining a present driving current of the lamp tube 21, establishing a directly proportional relationship of the present driving current and the illuminance of the UV light emitted by the lamp tube 21, determining whether to eliminate the difference between the illuminance in the housing 10 and the standard illuminance, if eliminating the difference between the illuminance in the housing 10 and the standard illuminance is determined, determining a value of increasing or decreasing the driving current, and increasing or decreasing the driving current of the lamp tube 21 according to the determined value.

The processor 60, based on the difference between the illuminance in the housing 10 and the standard illuminance and parameters of the lamp tube 21, may automatically adjust the illuminance of the UV light in the housing 10 when the illuminance of the UV light in the housing 10 is weak, to improve air purifying effect.

At block S97, determining whether an operating time of the lamp tube 21 is greater than a usage threshold value, if the operating time of the lamp tube 21 is greater than the usage threshold value, the procedure goes to block S98; if the operating time of the lamp tube 21 is smaller than or equal to the usage threshold value, the procedure ends.

The detected information may further include time information. The processor 60 may be electrically connected to the lamp tube 21, the processor 60 may start to time when the lamp tube 21 switches on and calculate a time every time the lamp tube 21 operates, to determine the operating time of the lamp tube 21.

The usage threshold value of the lamp tube 21 may be a normal usage time of the lamp tube 21 after the lamp tube 21 is made and tested by a usage time test.

At block S98, outputting usage time abnormal notification information.

The notification information may further include usage time abnormal notification information, the processor 60 may output the usage time abnormal notification information to the alarm device or the user equipment, which is not limited.

When the operating time of the lamp tube 21 is greater than the usage threshold value, the lamp tube 21 may be in danger if continuously operates, the processor 60 may output the usage time abnormal notification information to notice the user to replace the lamp tube 21.

In at least one embodiment, the processor 60 may output the usage time abnormal notification information to the user equipment to notice the user to replace the lamp tube 21. When the processor 60 outputs the usage time abnormal notification information to the user equipment, the usage time abnormal notification information may be in a form of e-mail, notification of applications, messages, etc.

A content of the usage time abnormal notification information outputted from the processor 60 to the user equipment may show the operating time of the lamp tube 21, the operating time of the lamp tube 21 may be obtained by the processor 60. The content of the usage time abnormal notification information outputted from the processor 60 to the user equipment may show text of lamp tube is overused or purification device is overused.

In at least one embodiment, the alarm device may output the alarm information in response to the usage time abnormal notification information. The alarm device may output different alarm information in response to the illuminance notification information and the usage time abnormal notification information. For instance, the alarm device may be alarm lights including a first alarm light and a second alarm light. When the alarm device receives the illuminance notification information, the first alarm light switches on; when the alarm device receives the usage time abnormal notification information, the second alarm light switches on.

In at least one embodiment, contents of the illuminance notification information and the usage time abnormal notification information may be in different forms. For instance, the processor 60 outputs the illuminance notification information and the usage time abnormal notification information to the user equipment in a form of e-mail, when the processor 60 outputs the illuminance notification information to the user equipment, the content of the illuminance notification information may be “purification device is abnormal”; when the processor 60 outputs the usage time abnormal notification information to the user equipment, the content of the usage time abnormal notification information may be “purification device is burn-in”.

In another embodiment, the illuminance notification information and the usage time abnormal notification information outputted to the user equipment may be in different forms. For instance, the processor 60 outputs the illuminance notification information to the user equipment in a form of e-mail and outputs the usage time abnormal notification information to the user equipment a form of application notification.

In at least one embodiment, after the processor 60 outputs the usage time abnormal notification information, and before the processor 60 determines whether the difference between the illuminance in the housing 10 and the standard illuminance is within the predetermined range in a next time point, that is before the block S93 in a next time point, the predetermined range may be adjusted, determining whether the processor 60 may improve the illuminance by adjusting the driving current through a predetermined range with a smaller maximum value, so as to decrease a danger of a burn-in processor 60 adjusting the driving current.

The blocks S91 to S98 may be continuous processing procedures, after block S98 ends, the processor 60 reoperates the blocks S91 to S98, to continuously detect and adjust the illuminance in the housing 10 and improve an air purifying stability.

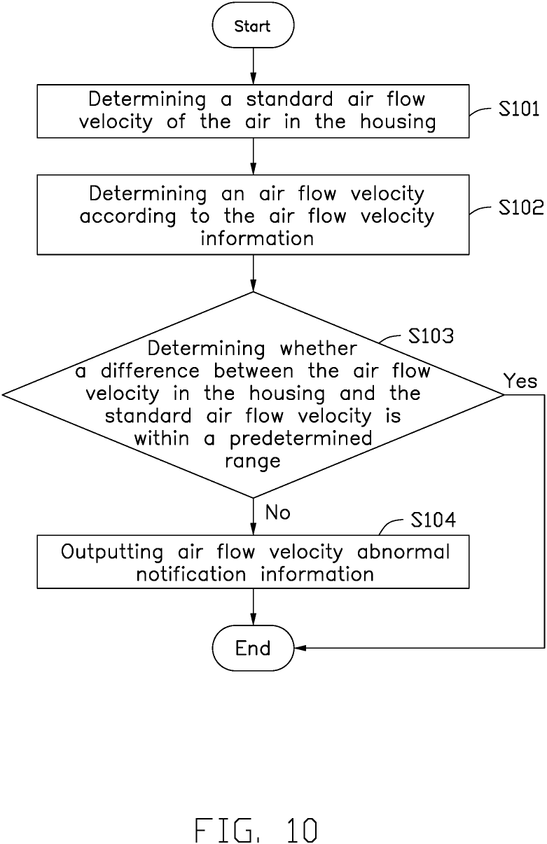

Referring to FIG. 10 illustrates another flow chat of at least one embodiment of the air purification method, the air purification method may be executed by the processor 60:

At block S101, determining a standard air flow velocity of the air in the housing 10.

The standard air flow velocity is corresponding to the air flow velocity of the air in the housing 10 when the air driving device 30 operates in a predetermined power. Since the predetermined power is known, the standard air flow velocity can be obtained.

In at least one embodiment, the processor 60 can be electrically connected to a data base (not shown), the processor 60 can obtain a working current value of the air driving device 30, the data base stores rated air flow velocity corresponding to different working current values, the processor 60 can determine a rated air flow velocity corresponding to the obtained working current value in the data base, the rated air flow velocity corresponding to the obtained working current value can be the standard air flow velocity. In other embodiments, the standard air flow velocity may be set by the user after detecting the working current value of the air driving device 30, such as by calculating or comparing the working current value to the rated air flow velocity in a specification of the air driving device 30, then the user can output the standard air flow velocity to the processor 60 through the user equipment, the processor 60 determines the rated air flow velocity as the standard illuminance, which is not limited.

At block S102, determining an air flow velocity according to the air flow velocity information.

The flow velocity detector 70 detects an air flow velocity in the housing 10 and generates air flow velocity detected information corresponding to the air flow velocity in the housing 10, the processor 60 obtains and analyzes the air flow velocity detected information to determine the air flow velocity in the housing 10.

At block S103, determining whether a difference between the air flow velocity in the housing 10 and the standard air flow velocity is within a predetermined range, if the difference is without the predetermined range, the procedure goes to block S104; if the difference is within the predetermined range, the procedure ends, the air flow velocity in the housing 10 is corresponding to the air flow velocity detected information.

When the difference between the air flow velocity in the housing 10 and the standard air flow velocity is within the predetermined range, the air flow velocity in the housing 10 is within a normal range, the purified air may be conveniently exported out of the housing 10. When the difference between the air flow velocity in the housing 10 and the standard air flow velocity is without the predetermined range, the air inlet 11 or the filter 15 being blocked is determined.

At block S104, outputting air flow velocity abnormal notification information.

When the difference between the air flow velocity in the housing 10 and the standard air flow velocity is great, outputting air flow velocity abnormal notification information to notice the user that the air inlet 11 or the filter 15 need cleaning.

In at least one embodiment, the processor 60 may output the air flow velocity abnormal notification information to the user equipment to notice the user that the air inlet 11 or the filter 15 need cleaning. When processor 60 may output the air flow velocity abnormal notification information to the user equipment, the air flow velocity abnormal notification information may be in a form of e-mail, notification of applications, messages, etc.

A content of the air flow velocity abnormal notification information outputted from the processor 60 to the user equipment may show the air flow velocity in the housing 10, the air flow velocity in the housing 10 may be generated by the processor 60 after analyzing the air flow velocity detected information. The content of the air flow velocity abnormal notification information outputted from the processor 60 to the user equipment may show text of “air flow velocity weak” or “filter blocked”.

In another embodiment, the processor 60 may be electrically connected to the alarm device, the alarm device outputs alarm information in response to the air flow velocity abnormal notification information. The alarm device may output different alarm information in response to the illuminance notification information, the usage time abnormal notification information, and the air flow velocity abnormal notification information. For instance, the alarm device may be alarm lights including a first alarm light, a second alarm light, and a third alarm light. When the alarm device receives the illuminance notification information, the first alarm light switches on; when the alarm device receives the usage time abnormal notification information, the second alarm light switches on; when the alarm device receives the air flow velocity abnormal notification information, the third alarm light switches on.

The blocks S101 to S 104 may be continuous processing procedures, after block S104 ends, the processor 60 reoperates the blocks S101 to S104, to continuously detect the air flow velocity in the housing 10.

Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. An air purification equipment comprising:
a housing, the housing defining an air inlet and an air outlet, the air inlet configured to import air into the housing, the air outlet configured to export air out of the housing;
a purification device arranged in the housing and configured to purify the air in the housing;
a guiding assembly arranged in the housing, the guiding assembly configured to guide the air from the air inlet to the purification device;
an air driving device arranged in the housing, the air driving device configured to drive the air from the air inlet to the purification device, and drive the air to the air outlet;
wherein the guiding assembly forms a first channel and a second channel, the first channel is corresponding to the air inlet, a cross-sectional area of the first channel is gradually decreasing along a direction from the air inlet towards the air outlet, a cross-sectional area of the of the second channel is same along the direction from the air inlet towards the air outlet, the first channel is configured to guide the air to flow into the second channel, at least a part of the purification device is positioned in the second channel.

2. The air purification equipment of claim 1, further comprising a light sensor, wherein the light sensor is arranged in the housing, the light sensor is configured to detect an illuminance of UV light in the housing emitted by the purification device.

3. The air purification equipment of claim 1, further comprising an air flow velocity detector, wherein the air flow velocity detector is arranged in the housing, the air flow velocity detector is configured to detect an air flow velocity in the housing.

4. The air purification equipment of claim 2, further comprising a processor, wherein the processor is electrically connected to the light sensor, the light sensor detects the illuminance of the light in the housing emitted by the purification device and outputs light detected information to the processor, the processor is configured to determine whether the purification device is abnormal according to the light detected information.

5. The air purification equipment of claim 4, wherein the processor is electrically connected to the air flow velocity detector, the air flow velocity detector detects the air flow velocity in the housing and outputs air flow velocity information to the processor, the processor is configured to determine whether the air inlet is blocked according to the air flow velocity information.

6. The air purification equipment of claim 1, wherein the guiding assembly comprises a surface arranged with a reflector, the reflector is configured to reflect the light emitted by the purification device.

7. The air purification equipment of claim 1, wherein the purification device comprises a lamp tube and a fiberglass sleeve, the lamp tube is configured to emit UV light, the fiberglass sleeve covers the lamp tube.

8. The air purification equipment of claim 1, wherein the guiding assembly further forms a third channel, the third channel and the first channel are positioned on opposite sides of the second channel, the third channel is corresponding to the air outlet, a cross-sectional area of the third channel is gradually increasing along the direction from the air inlet towards the air outlet, the third channel is configured to guide the air in the second channel to the air outlet.

9. The air purification equipment of claim 1, wherein the first channel and the second channel are both symmetric structures, the first channel is a funnel shaped, the second channel is a rectangular shaped.

10. The air purification equipment of claim 1, further comprising a filter, wherein the filter is secured to the air inlet, the filter is configured to block dust from the housing.

11. The air purification equipment of claim 4, further comprising a protection cover, wherein the protection cover is arranged in the housing, the protection cover covers the processor, the protection cover is configured to decrease an illuminance area of the light illuminates on the processor.

12. The air purification equipment of claim 7, wherein
the lamp tube is fully positioned in the second channel;
the lamp tube is partially positioned in the second channel, and the rest of the lamp tube is positioned in the first channel; or
the lamp tube is partially positioned in the second channel, and the rest of the lamp tube is extended out of the second channel from a direction away from the first channel.

13. The air purification equipment of claim 1, wherein a width of the second channel is smaller than a width of the housing, a cross-sectional area of the second channel is smaller than a cross-sectional area of the housing; a width of a portion of the first channel closes to the air inlet is smaller than or equal to the width of the housing, a cross-sectional area of the portion of the first channel closes to the air inlet is smaller than or equal to the cross-sectional area of the housing.

14. An air purification method applied in an air purification equipment, the air purification equipment comprises a housing, a purification device, a guiding assembly, and an air driving device, the housing defines an air inlet and an air outlet, the purification device, the guiding assembly, and the air driving device are arranged in the housing, the guiding assembly forms a first channel and a second channel, the air purification method comprising:
driving air from the air inlet to the first channel by the air driving device;
driving the air from the first channel to the second channel, a cross-sectional area of the first channel gradually decreasing along a direction from the air inlet towards the air outlet, a cross-sectional area of the second channel being smaller than the cross-sectional area of an end of the first channel closes to the air inlet;
purifying the air in the housing by the purification device, at least a part of the purification device being positioned in the second channel; and
driving the air from the second channel to the air outlet by the air driving device.

15. The air purification method of claim 14, wherein the guiding assembly further forms a third channel, the third channel and the first channel are positioned on opposite sides of the second channel, the third channel is corresponding to the air outlet, a cross-sectional area of the third channel is gradually increasing along the direction from the air inlet towards the air outlet, the air purification method further comprises:
driving the air from the second channel to the third channel by the air driving device; and
driving the air from the third channel to the air outlet by the air driving device.

16. The air purification method of claim 14, wherein the air purification equipment further comprises a light sensor, the light sensor is arranged in the housing, the air purification method further comprises:
determining a standard illuminance of UV light emitted by the purification device;
detecting an illuminance in the housing by the light sensor;
determining whether a difference between the illuminance in the housing and the standard illuminance is within a predetermined range;
if the difference between the illuminance in the housing and the standard illuminance being without the predetermined range is determined, outputting illuminance notification information.

17. The air purification method of claim 16, wherein the air purification method further comprises:
if the difference between the illuminance in the housing and the standard illuminance being within the predetermined range is determined, determining whether the difference between the illuminance in the housing and the standard illuminance is without a compensation range;
if the difference between the illuminance in the housing and the standard illuminance is without the compensation range, adjusting a driving current of the purification device to decrease the difference between the illuminance in the housing and the standard illuminance.

18. The air purification method of claim 17, wherein the air purification method further comprises:
determining whether an operating time of the purification device is greater than a usage threshold value; and
if the operating time of the purification device is greater than the usage threshold value is determined, outputting usage time abnormal notification information.

19. The air purification method of claim 17, wherein the air purification method further comprises:
if the difference between the illuminance in the housing and the standard illuminance is within the compensation range is determined, determining whether an operating time of the purification device is greater than a usage threshold value; and
if the operating time of the purification device is greater than the usage threshold value is determined, outputting usage time abnormal notification information.

20. The air purification method of claim 14, wherein the air purification equipment further comprises an air flow velocity detector, the air flow velocity detector is arranged in the housing, the air purification method further comprises:
determining a standard air flow velocity of the air in the housing when the air driving device operates in a predetermined power;
detecting an air flow velocity in the housing;
determining whether a difference between the air flow velocity in the housing and the standard air flow velocity is within a predetermined range; and
if the difference between the air flow velocity in the housing and the standard air flow velocity is without the predetermined range is determined, outputting air flow velocity abnormal notification information.
